(19) United States
(12) Patent Application Publication
(10) Pub. No.: US 2011/0015710 A1
(43) Pub. Date: Jan. 20, 2011

(54) CRIMP CONNECTION BETWEEN STIMULATION ELECTRODE AND CONDUCTION COIL

(75) Inventors: Christoph Vogt, St. Paul, MN (US); Lena Lewandrowski, Mainhausen (DE); Stefan Schibli, Frankfurt am Main (DE); Christiane Leitold, Woelfersheim (DE); René Richter, Babenhausen (DE)

Correspondence Address: DICKE, BILLIG & CZAJA, FIFTH STREET TOWERS, 100 SOUTH FIFTH STREET, SUITE 2250, MINNEAPOLIS, MN, 55402, US

(73) Assignee: W. C. HERAEUS GMBH, Hanau (DE)

(21) Appl. No.: 12/837,983

(22) Filed: Jul. 16, 2010

(30) Foreign Application Priority Data
Jul. 17, 2009 (DE) 10 2009 033 769.5

(51) Int. Cl. A61N 1/04; H01R 43/00

(52) U.S. Cl. 607/115; 29/854

(57) ABSTRACT

One aspect is a stimulation electrode including a base body. The base body encompasses a top area and an end area. The end area encompasses a groove-like, radially revolving coupling element, into which a connection area of a conduction coil can be pressed by means of plastic deformation.

### CROSS-REFERENCE TO RELATED APPLICATIONS

This Utility Patent Application claims priority to German Patent Application No. DE 10 2009 033 769.5, filed on Jul. 17, 2009, which is incorporated herein by reference. This Patent Application is also related to Utility Patent Applications filed on even date herewith, entitled “DETENT CONNECTION BETWEEN STIMULATION ELECTRODE AND CONDUCTION COIL” having Attorney Docket No. W683.105.101/P11135 US; entitled “CONNECTION ELEMENT FOR CONDUCTION COIL” having Attorney Docket No. W683.106.101/P11136 US; and entitled “CONNECTION BETWEEN STIMULATION ELECTRODE AND CONDUCTION COIL” having Attorney Docket No. W683.108.101/11154 US.

### BACKGROUND

One aspect relates to a stimulation electrode having a base body, wherein the base body encompasses a top area and an end area. One embodiment furthermore relates to a conduction coil for a medical electrode system. One embodiment furthermore relates to a medical electrode system having a conduction coil and a stimulation electrode, wherein the stimulation electrode encompasses a base body having a top area and an end area. One embodiment furthermore relates to a method for connecting a conduction coil to a stimulation electrode.

Stimulation electrodes as well as medical electrode systems are described in DE 10 2007 009 716 A1. Such stimulation electrodes must be connected to electric feed lines—also referred to as conduction coils. As a rule, the stimulation electrodes thereby consist of a high-melting metal, the feed lines of a metal having a lower melting temperature. These two components are often connected to one another by means of laser welding. However, due to the differences of the two metals, which are to be connected to one another, it is possible that the required mechanical stability or electric conductivity is not reached. Tears, which among others are caused by forming intermetallic phases or by the solidification behavior after the welding, can appear in the weld zone. Due to different melting temperatures, the fusion is partially insufficient. As a rule, such errors cannot be determined in a non-destructive manner, which can lead to considerable problems in the production or quality control, respectively.

For these and other reasons there is a need for the present invention.

### SUMMARY

One embodiment relates to a stimulation electrode having a base body, wherein the base body encompasses a top area and an end area. According to one embodiment, provision is made for the end area to encompass a groove-like, radially revolving coupling element, into which a connection area of a conduction coil can be pressed by means of plastic deformation.

### BRIEF DESCRIPTION OF THE DRAWINGS

The accompanying drawings are included to provide a further understanding of embodiments and are incorporated in and constitute a part of this specification. The drawings illustrate embodiments and together with the description serve to explain principles of embodiments. Other embodiments and many of the intended advantages of embodiments will be readily appreciated as they become better understood by reference to the following detailed description. The elements of the drawings are not necessarily to scale relative to each other. Like reference numerals designate corresponding similar parts.

Further advantages, features and details of embodiments result from the dependent claims and from the following description, in which a plurality of exemplary embodiments are described in detail with reference to the drawings. The features mentioned in the claims and in the description can thereby in each case be important for embodiments, either alone or in any combination.

FIG. 1 illustrates a stimulation electrode according to one embodiment.

FIG. 2 illustrates a schematic sectional enlargement of the stimulation electrode according to FIG. 1 for clarifying a coupling element.

FIG. 3 illustrates a further embodiment of the coupling element.

FIG. 4 illustrates a further embodiment of the coupling element.

FIG. 5 illustrates a conduction coil according to one embodiment.

FIG. 6 illustrates the conduction coil according to one embodiment having a deviating loop shape.

FIG. 7 illustrates a further conduction coil having a deviating loop shape.

FIG. 8 illustrates an alternative embodiment of the conduction coil.

FIG. 9 illustrates a further embodiment of the conduction coil according to one embodiment.

FIG. 10 illustrates an end area of the stimulation electrode having an engaged conduction coil.

FIG. 11 illustrates a sectional enlargement of an end area of the stimulation electrode according to one embodiment having an engaged conduction coil.

FIG. 12 illustrates a further embodiment of a medical electrode system.

FIG. 13 illustrates a further embodiment of the medical electrode system.

FIG. 14 illustrates an alternative embodiment of the medical electrode system.

FIG. 15 illustrates a medical electrode system analogous to FIG. 14 having an alternative coupling element.

FIG. 16 illustrates a further embodiment of the coupling element, clarified by means of a sectional enlargement of the medical electrode system.

FIG. 17 illustrates the medical electrode system from FIG. 16 having a crimp ring.

FIG. 18 illustrates a top view onto the medical electrode system according to FIG. 17.

FIGS. 19-21 illustrate the method steps in response to the connection of the conduction coil to the stimulation electrode.

FIG. 22 illustrates an electrode system having an attached crimp ring.

FIG. 23 illustrates the electrode system from FIG. 22 in a top view.

FIG. 24 illustrates a further embodiment of the electrode system having a crimp ring.

FIG. 25 illustrates a top view onto the medical electrode system from FIG. 24.

### DETAILED DESCRIPTION

In the following Detailed Description, reference is made to the accompanying drawings, which form a part hereof, and in which is shown by way of illustration specific embodiments in which the invention may be practiced. In this regard, directional terminology, such as “top,” “bottom,” “front,” “back,” “leading,” “trailing,” etc., is used with reference to the orientation of the Figure(s) being described. Because components of embodiments can be positioned in a number of different orientations, the directional terminology is used for purposes of illustration and is in no way limiting. It is to be understood that other embodiments may be utilized and structural or logical changes may be made without departing from the scope of the present invention. The following detailed description, therefore, is not to be taken in a limiting sense, and the scope of the present invention is defined by the appended claims.

It is to be understood that the features of the various exemplary embodiments described herein may be combined with each other, unless specifically noted otherwise.

According to one aspect of one embodiment, a stimulation electrode, a conduction coil and a medical electrode system, are created, in the case of which the mentioned disadvantages are avoided, and in the case of which a connection between the stimulation electrode and the conduction coil, which is stable and durable in the long run, is secured.

A stimulation electrode, a conduction coil, a medical electrode system, and a method for connecting a conduction coil having a stimulation electrode are proposed to solve this object. Features and details, which are described in context with the stimulation electrode or the conduction coil or the method, thereby also apply case in context with the medical electrode system and vice versa in each case.

For the stimulation electrode according to one embodiment, provision is made for the end area to encompass a groove-like, radially revolving coupling element, into which a connection area of a conduction coil can be pressed by means of plastic deformation.

To avoid the disadvantages occurring in response to connections between stimulation electrode and conduction coil by means of material engagement, a stimulation is proposed in the context of one embodiment, which encompasses a coupling element, into which a connection area of a conduction coil can be pressed by means of plastic deformation. The plastic deformation of the connection area leads to a positive connection between the stimulation electrode and the conduction coil. For this purpose, the conduction coil is pressed partially into the coupling element, which is cut into the end area in a groove-like manner. It is ensured with the plastic deformation of the conduction coil that the deformation is irreversible after the application of force has ended. Subsequent to the plastic deformation, a positive and/or non-positive connection between the coupling element of the stimulation electrode and the connection area of the conduction coil is thus created. This results in a non-positive and/or positive connection of the stimulation electrode to the conduction coil. This type of embodiment of the stimulation electrode, of the conduction coil and of the medical electrode system, which is formed from the two, provides for a connection without the disadvantages, which occur in response to connections by means of material engagement. The irreversible deformation of the conduction coil in the area of the coupling element results in an engaged connection between the coupling element and the conduction.

In the context of one embodiment, an irreversible, thus permanent deformation is identified as plastic deformation. This deformation remains even after the force, which causes the deformation, is removed. This permanent deformation is the remaining portion of a deformation, which goes beyond the elastic limit of a material. The plastic deformation is also identified as plastic strain or as plastic flow.

In the context of one embodiment, a recess within the end area of the coupling element, which is in one example is embodied in a cylindrical manner, is understood to be a groove and/or groove-like, radially revolving coupling element. Stimulation electrodes, which encompass dam-like structures in their end areas, are known in the state of the art. These dam-like structures rise above the end area and serve the purpose of connecting the stimulation electrode to a silicon tube, which shields said stimulation electrode. Contrary thereto, the stimulation electrode according to one embodiment differs in that the coupling element recesses into the end area. The width of the coupling element is small as compared to the width of the entire end area. It turned out to be advantageous in one embodiment when the width of the coupling element encompasses maximally 20% of the length of the end area. In the case of these length ratios, the conduction coil can be deformed securely into the coupling element and additionally also a further area—a part of the conduction area—of the conduction coil can be guided by the end area of the stimulation electrode.

According to one embodiment, provision is made for the stimulation electrode to encompass a coupling element, which revolves in the end area of the base body in a recessed manner. This coupling element acts like a clip closure, into which a part of the conduction coil—later referred to as connection area—is pressed in a positive and plastically deformed manner. This leads to a direct connection between the stimulation electrode and the conduction coil, without requiring a fusion and thus a connection by means of material engagement between both. In addition, the connection disclosed according to one embodiment can be determined in a non-destructive manner by means of the positive and plastically deformed introduction of the conduction coil into the coupling element, which thus leads to advantages in the production and quality control.

In the context of one embodiment, a “positive connection” refers to the engagement of at least two connection partners. In the case of the positive connection, the forces caused by the operating load are transferred at right angles to the two surfaces of the connection partners. According to one embodiment, a portion of the force, which is placed onto the stimulation electrode by means of the conduction coil or vice versa, can also be transferred in a non-positive manner. The description that stimulation electrode and conduction coil are connected to one another in a positive manner also does not eliminate an at least partial non-positive connection.

In the context of one embodiment, the term “stimulation electrode” does not refer to the transition point of the electric energy according to physical definition, but also refers to the technical line of electric conductor and can, if need be, also include an encasing insulation as well as all further functional elements, which are fixedly connected to the line. For clarification purposes, the section of the stimulation electrode, which actually operates in the physical sense and which includes the transition point of the electric energy, will be referred to hereinbelow as “electrically active surface.”

To further improve the connection between the conduction coil and the stimulation electrode, one embodiment of the stimulation electrode is characterized in that a bottom area of the coupling element is contoured so as to attain an at least area by area guiding of at least one loop of the conduction coil in the coupling element. For instance, the coupling element for example can encompass a rectangular cross section, whereas the conduction coil is wound of round wire in a helical manner. To attain the largest possible contact surface, a contour is introduced according to one embodiment into the bottom area of the coupling element. This contour can be embodied in a “V” or “W”-like manner. The width of each of the contours can thereby be adapted to the diameter of the wire from which the conduction coil is formed. In the context of one embodiment, the connection area of the conduction coil is pressed into the coupling element. Parts of the conduction coil can thereby also be pressed into the contour of the bottom area. In the context of the plastic deformation of the conduction coil, the wires of the conduction coil are pressed irreversibly into areas of the guides of the bottom area of the coupling element. This results in an increase of the contact surface between the coupling element and the loop as compared to non-contoured coupling elements. The cross section of the contour of the bottom area of the coupling element is adapted to the geometric shape of the conduction coil and/or of the loops of the conduction coil. An improvement of the transmission of force—in a positive as well as non-positive manner—can thus be attained between the conduction coil and the coupling element by increasing the contact surface between the coupling element and the conduction coil.

A further embodiment of the stimulation electrode is characterized in that the bottom area of the coupling element encompasses a sawtooth-like cross section. In this case, the bottom area does not run substantially parallel to the end area of the stimulation electrode. Instead, the coupling element is recesses in the direction of the end of the stimulation electrode. The cross section of the coupling element thus attains a sawtooth-like course. This course provides for a self-locking of the conduction coils in the radially revolving coupling element. When pulling the conduction coil with a force, the result is that the conduction coil is pulled farther into the coupling element and can thus better accommodate the force. By pressing the connection area into the coupling element, which is embodied in a sawtooth-like manner, the course of the connection area adapts to the sawtooth-like course of the bottom area. The individual loops of the connection area consequently encompass different diameters, after they have been pressed into the coupling element by means of plastic deformation. This also facilitates the force transition between conduction coil and stimulation electrode.

A further embodiment of the stimulation electrode is characterized in that the stimulation electrode encompasses at least one from the group tantalum, niobium, titanium or platinum, and in one embodiment, that the stimulation electrode encompasses a TaNbW alloy. The mentioned group of metals is characterized by a biocompatibility as well as by a high electric conductivity. In one embodiment, the stimulation electrode encompasses a tantalum-niobium-tungsten alloy (TaNbW alloy including 10 weight % of niobium and 7.5 weight % of tungsten) or consists thereof. In one embodiment, the tantalum-niobium-tungsten alloy turned out to be preferred as base material for the base body and the tantalum oxide layer, because its tensile strength is almost twice as high and because its specific capacitance is almost twice as high as compared to that of the often used platinum-iridium-10 alloy (PtIr10). A reduction of the losses in response to the transfer of stimulation pulses is thus possible.

The stimulation electrode according to one embodiment can encompass the valve metal tantalum or can consist thereof. A stimulation electrode, which is embodied in such a manner, can be provided with a tantalum oxide layer by means of high voltage pulses. The method used for this is also referred to as plasma-electrolytic oxidation (PEO) and is described in more detail in WO 2006/104432 A1 for niobium. In the disclosed method, a porous structure of the corresponding metal oxide is generated on the surface of the stimulation electrode by means of plasma-electrolytic oxidation. It thereby turned out to be an anomaly that the porous structure encompasses pores, which are considerably larger than it is known from the current state of the art. This thus leads to a stimulation electrode including an electrically conductive base body, wherein the base body, for example, encompasses tantalum and the base body is at least partially covered with a porous tantalum oxide layer, which is anodically applied by means of high voltage pulses.

One embodiment also relates to a conduction coil for a medical electrode system. For conduction coils from the state of the art, disadvantages have already been described above. According, one embodiment discloses a conduction coil, which provides for a connection to a stimulation electrode, which is stable and durable in the long run. In the context of one embodiment, a conduction coil for a medical electrode system is disclosed including a conduction area and a connection area, wherein the connection area is connected to the conduction area and the connection area can be pressed into a coupling element of a stimulation electrode by means of plastic deformation. It goes without saying that features and details, which have already been described in context with the stimulation electrode and which refer to the conduction coil, also apply to the conduction coil according to one embodiment disclosed herein and vice versa.

The core of one embodiment is that the conduction coil is embodied such that it provides for a plastic deformation of at least a section, which is then pressed into the coupling element. This anomaly can be attained in that the entire conduction coil is made from a material, which provides for a plastic deformation. In the alternative, only the connection area can be produced from a material, which can be plastically deformed. It is possible in the case of this embodiment alternative for the conduction area to either be brittle and thus not plastically deformable or for it to consist of a material, in the case of which a larger force is required for a plastic deformation than in the case of the material of the connection area.

The loops of the conduction coil can be embodied as multiple coils, wherein the individual wires can be located coaxially and/or parallel to one another and can encompass the same outer diameter. The loops of the conduction coil can also be wound in a multifilar manner from a plurality of wires and can be provided with an electric insulation.

A further embodiment of the conduction coil is characterized in that the conduction coil encompasses at least one from the group MP-35, MP-35N and DFT. In this alternative, the conduction coil can encompass a “drawn filled tube” (DFT). Such DFTs encompass two components, a bio-resistant, biocompatible and non-toxic component and a component of a material including a low electric resistance. For the most part, the bio-resistant, biocompatible and non-toxic component is embodied to protect the component of a material including a low electric resistance. Platinum, iridium or an alloy of these two materials is preferred in one embodiment. In a further embodiment, the core including a low electric resistance consists of a material from the vanadium group (5^{th} subgroup of the periodic table of the elements) or from the copper group (1^{st} subgroup of the periodic table of the elements). In one embodiment, the core of the DFT wire consists of tantalum (Ta), niobium (Nb) or gold (Au). The conduction coil can furthermore encompass MP-35 and/or MP-35N (MP35N is a protected mark of SPS Technologies, Inc.). MP35N substantially encompasses approximately 35 weight % of nickel, approximately 35 weight % of cobalt, approximately 20 weight % of chromium and approximately 10 weight % of molybdenum.

In one embodiment, to attain an advantageous mass ratio for the conduction coil, which forms a defibrillation electrode, provision can be made for an outer diameter of this conduction coil to be at least five, six or seven times the diameter of the coil-forming wire or an intermediate value thereof. This leads to a flexible conduction coil including an advantageous outer dimension, which provides for a sufficiently large field intensity distribution in response to the defibrillation and thus for a relatively low shock energy.

A further embodiment form of the conduction coil is characterized in that the connection area includes one or a plurality of loops of the conduction coil. The loops are arranged in a helical manner, so as to form the conduction coil. In an embodiment, a plurality of at least two loops is connected by means of material engagement, for instance by means of welding. The connection of the loops by means of material engagement leads to a type of connection element, which engages with the groove-like coupling element in a positive manner. By means of the connection of a plurality of loops of the conduction coil in the connection area, it is furthermore avoided that the respective wire ends and/or loop ends of the conduction coil unwind when in use.

One embodiment also relates to a medical electrode system including a conduction coil and a stimulation electrode, wherein the stimulation electrode encompasses a base body including a top area and an end area. Some disadvantages for a medical electrode system, which is embodied in such a manner, have already been described above. This also results in the above-specified object. To solve it, provision is made according to one embodiment for the end area to encompass a groove-like, radially revolving coupling element, the conduction coil to encompass a connection area on the end side and for the connection area to be engaged with the coupling element in a positive manner after a plastic deformation. The medical electrode system according to one embodiment is composed of the stimulation electrode according to one embodiment and of the conduction coil according to one embodiment. The two elements—stimulation electrode and conduction coil—are thereby adapted to one another such that, in combination, they form the medical electrode system according to one embodiment. The conduction coil is pressed into the coupling element of the stimulation electrode by means of the plastic deformation of the connection area thereof. This results in a positive engagement. In a connection situation, the connection area of the conduction coil is thus plastically molded into the coupling element. It goes without saying that features and details, which have been described with reference to the conduction coil or the stimulation electrode, also apply to the medical electrode system and vice versa.

The medical electrode system serves as electric connection between an electrotherapeutic, implantable apparatus, which can be a neuro stimulator, a pace maker, a defibrillator or another suitable electrotherapeutic implantable apparatus and the area in the body, which is to be treated. These areas in the body can be of the most varying type, such as a heart, for example. The medical electrode system cannot only serve to transfer therapeutic pulses, but also to transfer body and measuring signals to the implant, so that suitable therapy can specifically be performed in answer to the body signals.

The medical electrode system according to one embodiment encompasses an elongate body including a proximal and a distal end. Provision is made on the proximal end for a connection to an electrotherapeutic implantable apparatus. This can be a pace maker, a cardioverter/defibrillator or another suitable heart rhythm apparatus. A fastening device for securely fastening the stimulation electrode to the cardiac tissue is located at the distal end. On the one hand, this can be a so-called passive fixation, which is embodied in an anchor-shaped manner and which can thus hook into the myocardial muscle. On the other hand, it can be an active fixation, which can actively be screwed into the cardiac tissue by means of a helical screw, which can be screwed in. This helically embodied stimulation electrode can also be electrically conductive and can thus act as additional electrically active surface. The area of the medical electrode system, which is located between the proximal and distal end, can furthermore be sealed and insulated from the environment. The outer surface is thereby coated with silicon or a similar synthetic material. In the distal area of the medical electrode system, the sealed and insulated outer surface is interrupted by at least one electrically active surface. These electrically active surfaces are areas of the stimulation electrode, which can provide for a stimulation of the above-mentioned type in the atrium of the heart, for example.

An embodiment of the medical electrode system is characterized in that the two parts conduction coil and stimulation electrode, which are connected to one another, are formed from metals including different melting temperatures from the group consisting of the elements Pt, Pd, Ag, Au, Nb, Ta, Ti, Zr, W, V, Hf, Mo, Co, Cr, Ni, Ir, Re, Ru as well as from alloys on the basis of at least one of these elements, and in one embodiment, that the metal of the conduction coil encompasses a lower melting temperature than the metal of the stimulation electrode. It is known in the state of the art to connect stimulation electrodes and conduction coil to one another by means of welding, for example, laser welding. However, difficulties arise due to the differences, for example, in the melting temperatures of the two materials, which are to be connected to one another. For instance, tears can appear in the melting zone, which are caused by the different solidification behavior of the two used materials.

The medical electrode system according to one embodiment lends itself so as not to have to do without the use of materials including very different melting temperatures for the conduction coil on the one hand and for the stimulation electrode on the other hand. By means of the described embodiment of the conduction coil and of the stimulation electrode—which result in the medical electrode system according to one embodiment—the conduction coil on the one hand and the stimulation electrode on the other hand can in each case be formed from metals, which encompass very different melting temperatures. In one embodiment alternative, the difference of the melting temperatures of the two parts—conduction coil and stimulation electrode—is at least 1000° C., and in one example, at least 1500° C. Another embodiment alternative is characterized in that the melting temperature of the higher melting material is at least 2400° C., and in one example, at least 2800° C. In an embodiment alternative, the conduction coil can encompass MP-35, for instance, and the stimulation electrode can encompass tantalum. MP35 (approximately 35 weight % of nickel, approximately 35 weight % of cobalt, approximately 20 weight % of chromium and approximately 10 weight % of molybdenum) has a melting point of approximately 1400° C. Tantalum has a melting point of 2996° C., so that the temperature difference of the melting temperatures of the two materials is greater than 1500° C. In a further example, the stimulation electrode can be formed from Ta-10W, including a melting point of 3040° C. The conduction coil is formed from a core jacket wire (DTF), wherein the core consists of tantalum and the jacket consists of MP35N. Here, the difference of the melting temperatures of the two parts is above 1500 ° C. as well. In a further example, a niobium-containing stimulation electrode is used as stimulation electrode. In the event that a conduction coil of MP35N is used, the difference of the melting temperatures is more than 1000° C., because the melting temperature of niobium is 2468° C. However, a direct connection of the two parts conduction coil and stimulation electrode is possible without disadvantages with a stimulation electrode and conduction coil embodied according to one embodiment, because a material connection is not desired.

A further embodiment of the medical electrode system according to one embodiment is characterized in that a crimp ring presses the connection area into the coupling element at least area by area. Provision is thereby made for the crimp ring as well as for the connection area to be plastically deformed. The crimp ring can thereby at least partially flow into areas of the connection area and can thus possible lead to a connection by means of material engagement and/or a non-positive and/or positive connection between crimp ring and connection area. The crimp ring reinforces the connection between the conduction coil and the stimulation electrode. The crimp ring can thereby be composed of the materials of the conduction coil and/or of the stimulation electrode.

In a further embodiment, the medical electrode system is characterized in that the connection area engages with the coupling element in a positive manner after a plastic deformation of the crimp ring. In this embodiment alternative, the crimp ring is plastically deformed. The connection area of the conduction coil can, but must not, be plastically deformed. The plastic deformation of the crimp ring ensures that the connection area is pressed into the coupling element such that a positive connection between the connection area and the coupling element is created. The crimp ring ensures that the connection area cannot disengage from the coupling element. Instead, the crimp ring ensures that the loops of the connection care are pressed into the radially revolving coupling element. In a connection situation—thus in the case of a positive connection between conduction coil and stimulation electrode—the crimp ring is plastically molded into the coupling element at least area by area such that the connection area engages with the coupling element in a positive manner.

The use of a crimp ring for connecting a conduction coil and a stimulation electrode is furthermore claimed in the context of one embodiment. As is presented above, the crimp ring can be used to press the connection area of the conduction coil into the coupling element of the stimulation electrode. The use of a crimp ring, which is characterized in that the crimp ring plastically deforms a connection area of the conduction coil, is claimed in a further embodiment. In one embodiment, it furthermore turned out to be advantageous when the crimp ring encompasses at least one from the group Ta, Ti, Nb or Pt, and in one example, when the crimp ring encompasses MP-35. It goes without saying that features and details, which have been described in context with the conduction coil, the stimulation electrode and the medical electrode system, also apply to the use of the crimp ring in context with the mentioned parts.

One embodiment also relates to a method for connecting a conduction coil to a stimulation electrode. Disadvantages resulting in the case of known methods have already been described above. This results in the object, which is also mentioned above. Provision is made according to one embodiment for a connection area of the conduction coil to be pressed into a groove-like coupling element of the stimulation electrode by means of a plastic deformation. In the context of one embodiment, the term of the plastic deformation mentioned herein also includes that the connection area of the conduction coil is plastically deformed as well as that only one crimp ring, which is placed over the connection area, is plastically deformed, without resulting in a plastic deformation of the connection area.

It goes without saying that features and details, which have otherwise been described for the conduction coil, the stimulation electrode or the medical electrode system, also apply in context with the method according to one embodiment and vice versa.

One embodiment of the method is characterized in that the connection area of the conduction coil is pressed into a groove-like coupling element of the stimulation electrode by means of a crimp ring. In one embodiment, an advantage is thereby given when the loops of the connection area and/or an end of the conduction coil are welded, so as to prevent an unwinding. It turned out to be advantageous in one embodiment when at least two loops of the connection area are welded at least area by area. This welding also prevents an unwinding of the loops of the conduction coil in the connection area.

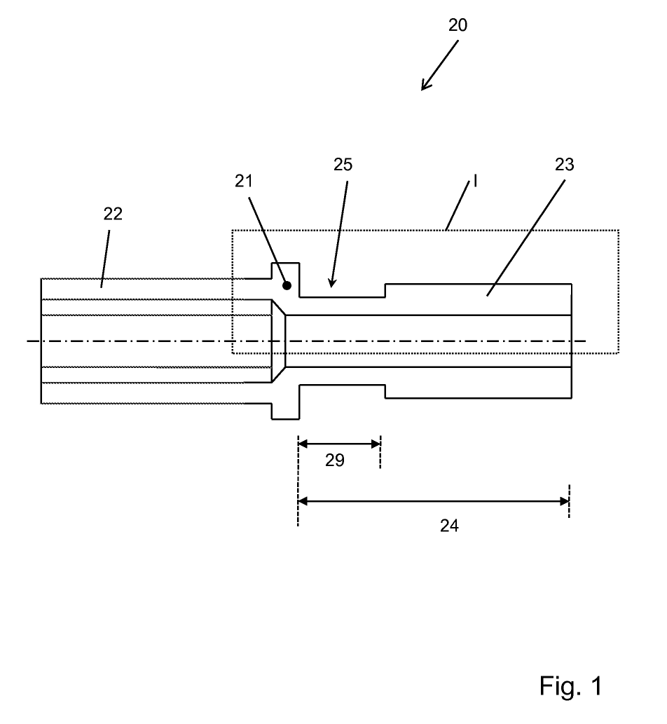

A stimulation electrode 20 according to one embodiment is illustrated in FIG. 1. The stimulation electrode 20 encompasses a cylindrically embodied base body 21. The base body 21 encompasses a top area 22 and an end area 23. The illustration of the top area 22 is only schematic. The top area 22 encompasses an active surface, which is used to transfer and/or to sense electric pulses. The base body 21 furthermore encompasses the end area 23, which is also embodied cylindrically. A borehole runs in the interior of the base body 21, so that the stimulation electrode is embodied in a tube-like manner. The end area 23 encompasses a groove-like radially revolving coupling element 25, into which a connection area 42 of a conduction coil 40 can be pressed and/or a crimp ring. In the illustrated exemplary embodiment, the coupling element 25 encompasses a rectangular cross section. End areas, which encompass dam-like superstructures, which are used to connect a stimulation electrode to a silicon tube, are known in the state of the art. Contrary thereto, the coupling element 25 is a recess, which is introduced into the material of the end area 23. The term “groove-like” refers to the fact that the recessed surface of the coupling element 25 is smaller than the total surface of the cylindrically embodied end area. In the case of a dam-like structure, the projection dams encompass a surface, which is smaller than the areas of the end area 23 located therebetween. It turned out to be advantageous in one embodiment when a width 29 of the coupling element 25 is maximally 25% of the length 24 of the end area 23.

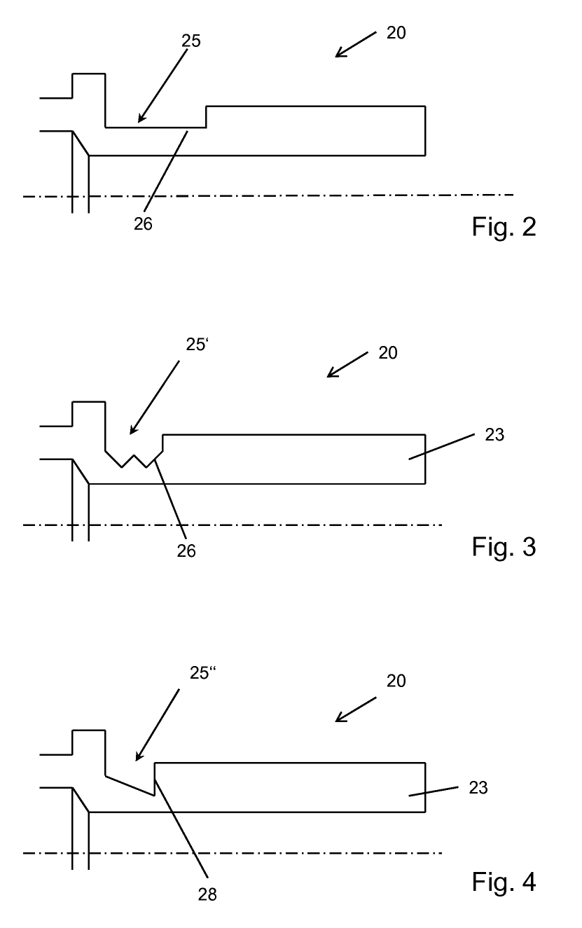

A sectional enlargement of a stimulation electrode 20, which is embodied according to one embodiment, is in each case illustrated in FIGS. 2-4 and 11-16 below. The sectional enlargement thereby includes that area, which is identified in Figure with the character I. FIG. 2, which illustrates the sectional enlargement of the stimulation electrode 20 illustrated in FIG. 1, clarifies this. This is a stimulation electrode 20 including a coupling element 25, which is embodied in a rectangular, groove-like manner and which revolves around the periphery of the base body 21 in radial direction.

FIGS. 3 and 4 illustrate the area I of the stimulation electrode 20. In FIG. 3, the stimulation electrode 20 encompasses a contoured coupling element 25′. A bottom area 26 of the coupling element is thereby contoured, so as to attain a guiding of at least one loop of a conduction coil in the coupling element 25 at least area by area. In the illustrated exemplary embodiment, the bottom area 26 encompasses a “V”-like embodiment. The bottom area 26 is provided with two V-shaped cuts, in which a loop of a conduction coil can in each case be supported. The support of a loop of a conduction coil 40 in the V-shaped bottom area 26 leads to a larger contact surface between the conduction coil and the coupling element 25 or the stimulation electrode 20, respectively. An improved transmission of force is thus made possible between the stimulation electrode 20 and a conduction coil 40.

FIG. 4 illustrates a bottom area 26 of the coupling 25″, which encompasses a sawtooth-like cross section. The coupling element 25″ thus recesses in the direction of an end of the end area 23. This recessed bottom area 26 of the coupling element 25″ makes sure that the conduction coil is pulled in the direction of a wall area 28 in response to a stress.

The coupling element 25, 25′, 25″ of the stimulation electrode 10 according to one embodiment is in each case designed such that the connection area of a conduction coil is pressed into it. According to one embodiment, the pressing is to take place by means of a plastic deformation. In a first step, it is to be understood thereby that the connection area 42 of the conduction coil 40 is plastically deformed. This plastic deformation leads to an irreversible change of the radius of the conduction coil 40, so that the conduction coil or the connection area, respectively, can no longer be removed from the coupling element. In a second embodiment alternative, which will be described in detail below, a crimp ring is plastically deformed and thereby presses the connection area 41 of the conduction coil 40 into the radially revolving coupling element 25, 25′, 25.″ A plastic deformation of the connection area 41 can, but must not, take place in this case. Consequently, three possible plastic deformations are claimed in the context of one embodiment:
a plastic deformation of the connection area 41 of the conduction coil 40,
a plastic deformation of a crimp ring 60 and
a plastic deformation of the connection area 41 and of the crimp ring 60.

The coupling element 25, 25′, 25″ is thereby designed such that it ensures in each case a positive connection to the connection area 42 of the conduction coil 40 independent on the plastically deformed component, which is to be used.

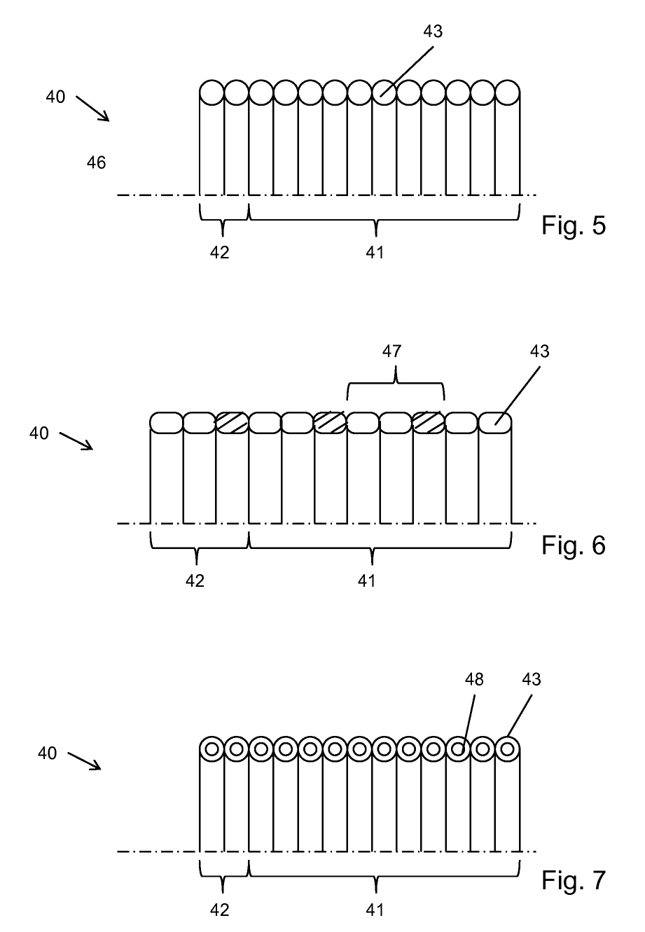

FIG. 5-9 clarify a plurality of conduction coils 40, which are embodied according to one embodiment, for a medical electrode system 10. The conduction coils 40 serve as transport lines for electric pulses from a body part to a medically implantable apparatus or in opposite direction. The conduction coil 40 thereby encompasses a conduction area 41 and a connection area 42. The conduction coil 40 can be connected to the stimulation electrode 20 by means of the connection area 42. The connection area 42 connects to the conduction area 41 on the end side. The starting point for the embodiment according to one embodiment of the stimulation electrode 20 and of the conduction coil 40 are problems resulting in response to the connection of two material by means of material engagement, which encompass very different melting temperatures. A different solidification behavior results in tears and intermetallic phases, which lead to a weakening of the connection. To overcome this disadvantage, the conduction coil 40 according to one embodiment encompasses a connection area 42, which is used to be pressed into the groove-like coupling element 25.

FIG. 5 clarifies that the conduction 40 consists of individual loops 43, which encompass a circular cross section. To form the conduction coil and/or the loops 43 of the conduction coil, a wire is wound in a helical manner. It is also possible for the loops 43 of the conduction coil 40, which are formed by the wire, to encompass a rectangular cross section, as is clarified in FIG. 6. This cross section can be generated, for example, by pulling or crushing a wire, which is otherwise provided with a round cross section. FIG. 6 is to furthermore clarify that the conduction coil 40 can be formed by a plurality of wires. In this case, three helical parts are screwed into one another such that a conduction coil 40 is created, in the case of which the individual loops 43 of the helical parts are arranged next to one another. This is to be clarified by means of shading, which illustrates one of the three helical parts. The three helical parts, which form the conduction coil 40, thereby form loop packets 44, which include three loops 43 in each case.

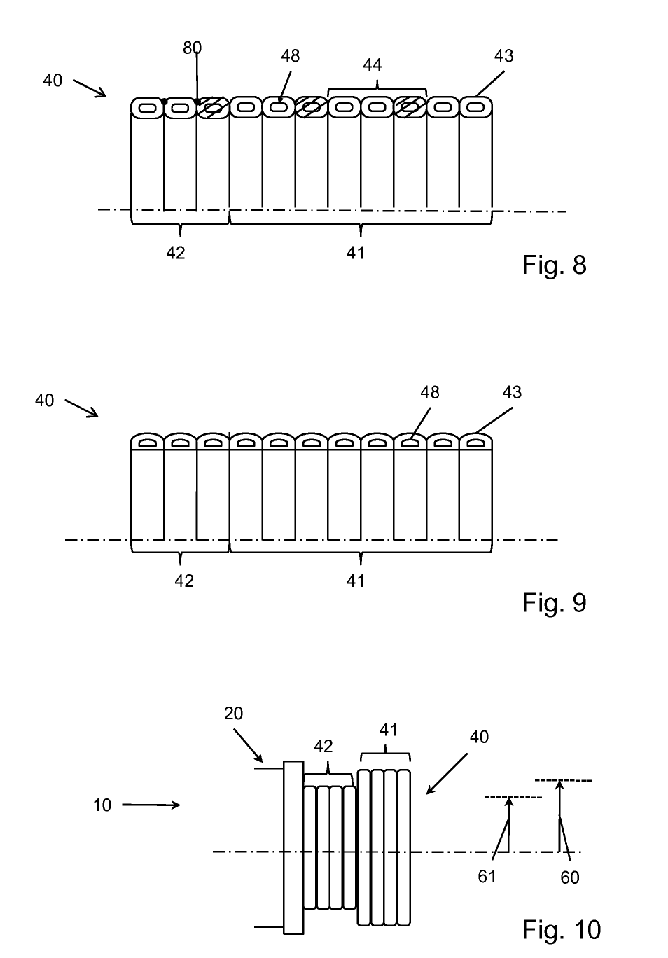

FIGS. 7-9 illustrate further exemplary embodiments of the conduction coil 40. The used conduction coils 40 are so-called “drawn filled tubes” (DFT). A DFT thereby encompasses two components: a bio-resistant, non-toxic component and a component consisting of a material including a low, electric resistance. Generally, the bio-compatible component forms a cover over the component including the low electric resistance. In FIGS. 7-9, this is to be clarified in that each of the loops 43 encompasses a core 48, which is to represent the component including the low electric resistance. As is clarified in the figures, the cross section of the individual loops 43 can vary. FIG. 7 illustrates a round cross section, FIG. 8 illustrates a rectangular and FIG. 9 a flattened cross section. Depending on the designated use, one of the illustrated types of the conduction coil 40 can be used. FIG. 8 thereby clarifies again the use of helical parts, which are twisted into one another several times, for forming the conduction coil 40, which results in loop packets 44. The loops of the loop packet 44 were furthermore connected to one another in the connecting area 42 by welding points 80 at least area by area by means of material engagement. This connection by means of material engagement prevents the loops 43 and/or loop packets 44 from unwinding and/or from becoming fibrous.

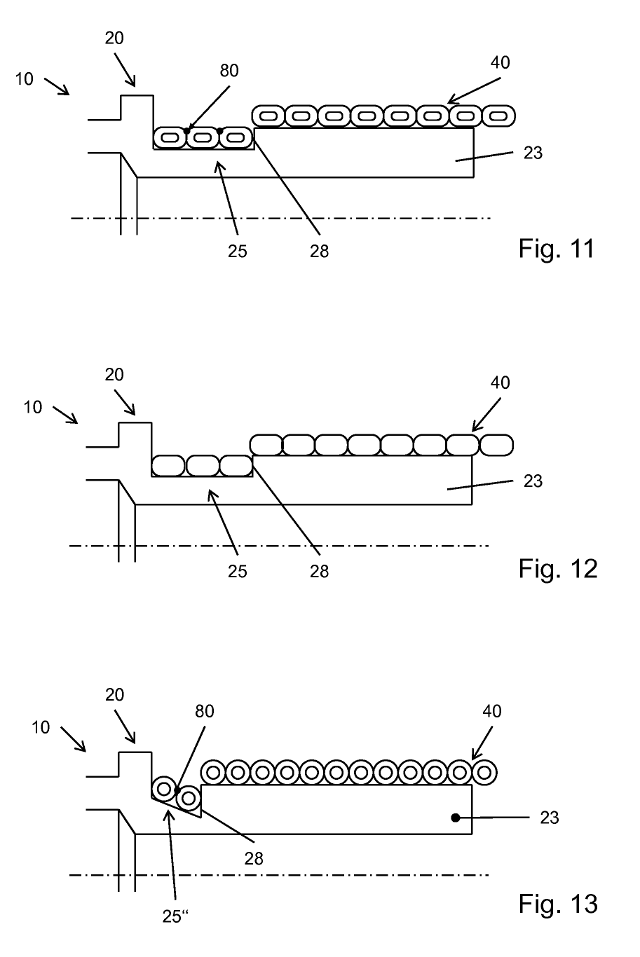

FIG. 10 illustrates an end area of a stimulation electrode 20. According to one embodiment, the connection area 42 of the conduction coil 40 was pressed into the coupling element by means of plastic deformation. Due to the plastic deformation of the connection area 42, the first radius 61 thereof is smaller than a second radius 60 of the conduction area 41 of the conduction coil 40, which is not plastically deformed. A cascaded embodiment of the conduction coil 40 thus results after the connection—thus after the plastic deformation of the connection area 42. FIGS. 11-16 below are to also clarify this.

FIGS. 11-16 illustrate the medical electrode system 10 according to one embodiment. The area I from FIG. 1 of the stimulation electrode 20 including the conduction coil 40, which is slid on and which engages with the coupling element 25, is illustrated in each case. FIG. 11 illustrates a medical electrode system 10 including a stimulation electrode 20, which encompasses a rectangular, groove-like, radially revolving coupling element 25. FIG. 11 also illustrates a connection situation. In this connection situation, the connection area 42 of the conduction coil 40 engages with the coupling element 25 in a positive manner after a plastic deformation. This thus leads to a positive connection between the conduction coil 40 and the stimulation electrode 20.

In FIGS. 11-16, the connection area 42 of the conduction coil 40 was molded into the coupling element by means of plastic deformation. It can be seen that the connection area 42 comes to rest in the coupling element 25 in each case so as to result in a positive connection. The connection area 42 and thus the conduction coil 40 are engaged with the coupling element 25, 25′, 25″ in a positive manner. In this position as well as in the position identified as connection situation, the radius of the connection area has been shrunk irreversibly, so as to establish a connection between the conduction coil 40 and the stimulation electrode 20 and so as to form the medical electrode system 10 according to one embodiment. The conduction coil 40 and the stimulation electrode 20 encompass in each case metals including different melting temperatures. The type of the embodiment and connection described herein turned out to be advantageous herein, for example, for such elements of a medical electrode system 10—conduction coil 40 and stimulation electrode 20, each of which encompass very different melting temperatures. By avoiding a connection between conduction coil 40 and stimulation electrode by means of material engagement, possible problems cannot arise in response to the melting of the individual connection partners. The non-positive and/or positive connection described herein, which is created by means of a plastic deformation of the connection area 42 and/or of a crimp ring 60, which is to be described below, overcomes the afore-mentioned disadvantages.

The conduction coil 40 can introduce forces into the stimulation electrode via this positive connection. FIGS. 11 and 12 differ in each case only in the embodiment of the conduction coils 40. FIG. 11 illustrates a conduction coil 40 of DFTs, wherein the loops 43, which are supported within the coupling element 25, form a loop packet 44 by means of connections by means of material engagement.

FIG. 13 illustrates a stimulation electrode 20, the coupling element of which encompasses a sawtooth-like bottom area 26. This coupling element 25″ reinforces the positive connection between the stimulation electrode 20 and the conduction coil 40. In the event that a tractive force is introduced into the medical electrode system 10, in response to which the conduction coil is pulled away from the stimulation electrode 20, the loops 43 push against a wall area 28 of the coupling area 25″ in the area of the coupling element 25.” The sawtooth-like cross section of the bottom area 26 of the coupling element 25″ leads to a self-locking of the loops within the coupling element 25.” This avoids the danger that, due to an application of force, possible loops can jump out of the coupling element 25″ when force is applied to the conduction coil.

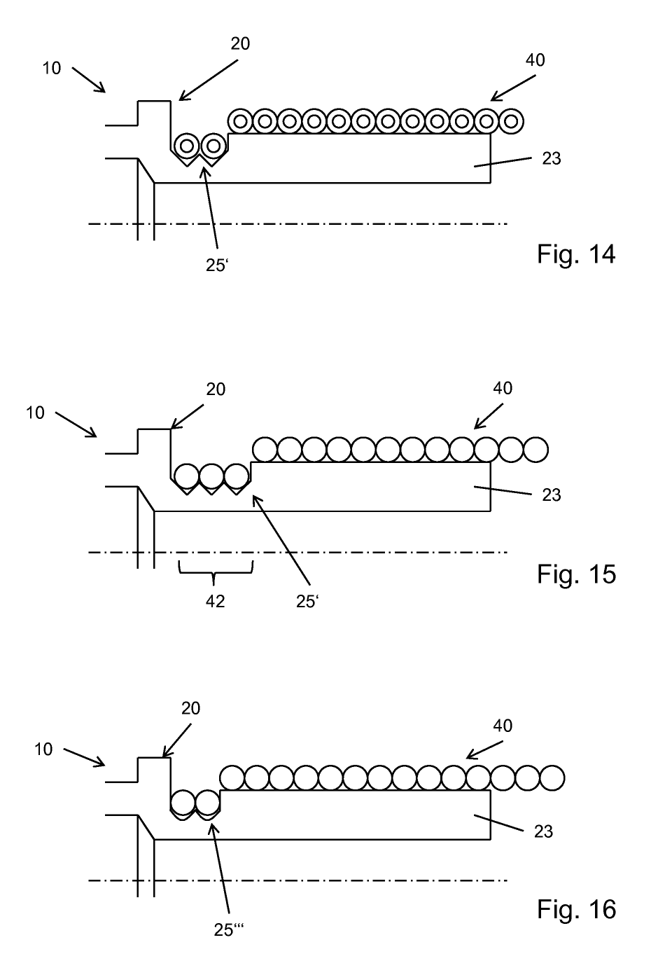

A further embodiment of the medical electrode system 10 according to one embodiment is characterized in that a bottom area 26 of the coupling element 25 is contoured so as to attain a guiding of at least one loop of the conduction coil 40 in the coupling element, at least area by area. This is clarified in FIGS. 14-16. They illustrate the area I of the stimulation electrode 20 including the conduction coil 40, which is pressed in in a positive manner. In FIGS. 14 and 15, the bottom area 26 is embodied in “V”-like manner. The coupling element 25′ thus provides for a guiding of the loops 43 of the connection area 42. In FIG. 14, the contour encompasses two grooves, which are embodied in a “V”-like manner and in which a loop rests in each case. The width of the respective contour is a function of the diameter and of the embodiment of the individual loops 43 of the conduction coil 40. In FIG. 15, the coupling element encompasses three “V”-like contours in the bottom area 26. Depending on the form, it can also be advantageous when a “W”-like contour of the bottom area is used, as it is illustrated in FIG. 16. The bottom area 26 is thereby provided with recesses, which are designed so as to be round. The coupling element 25″′ resulting in this manner can be advantageous in the case of conduction coils 40 including loops 43, which encompass a circular cross section.

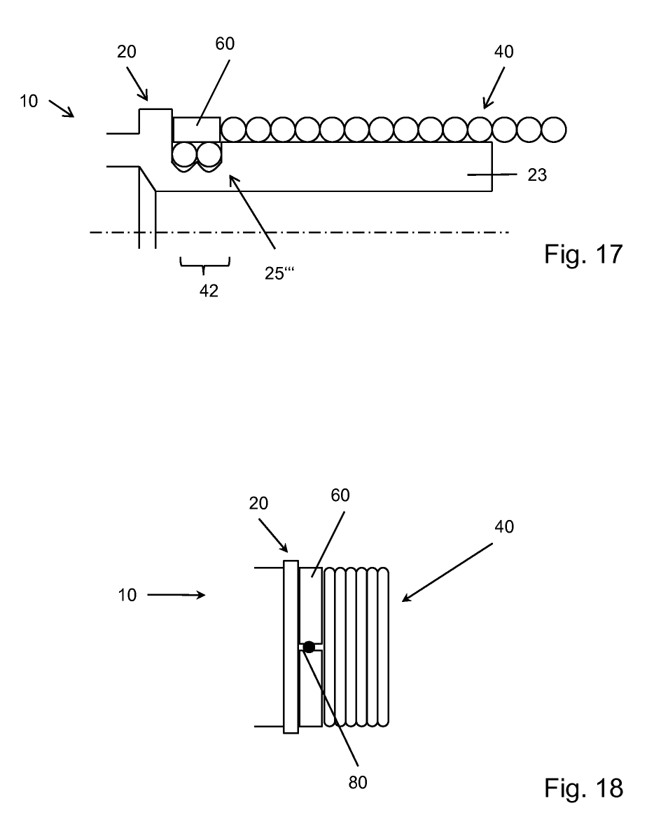

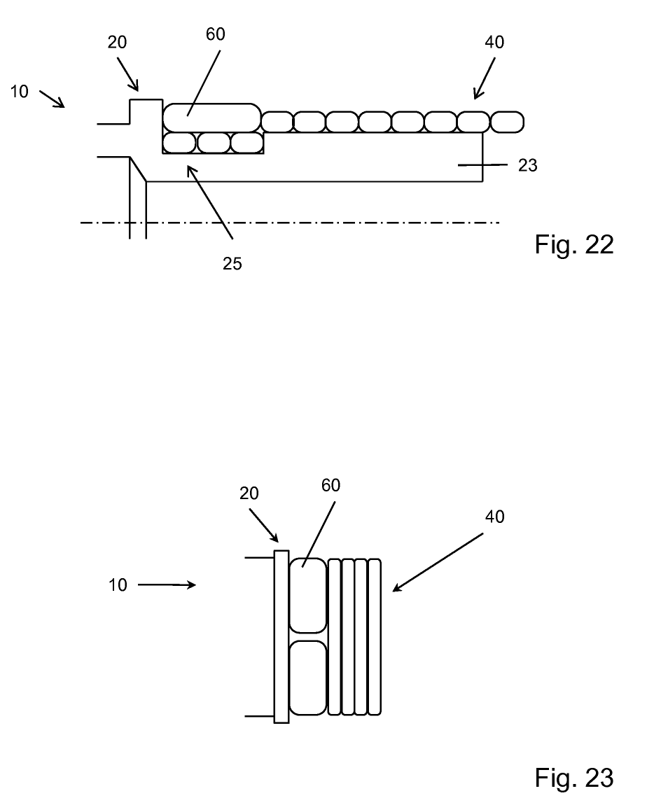

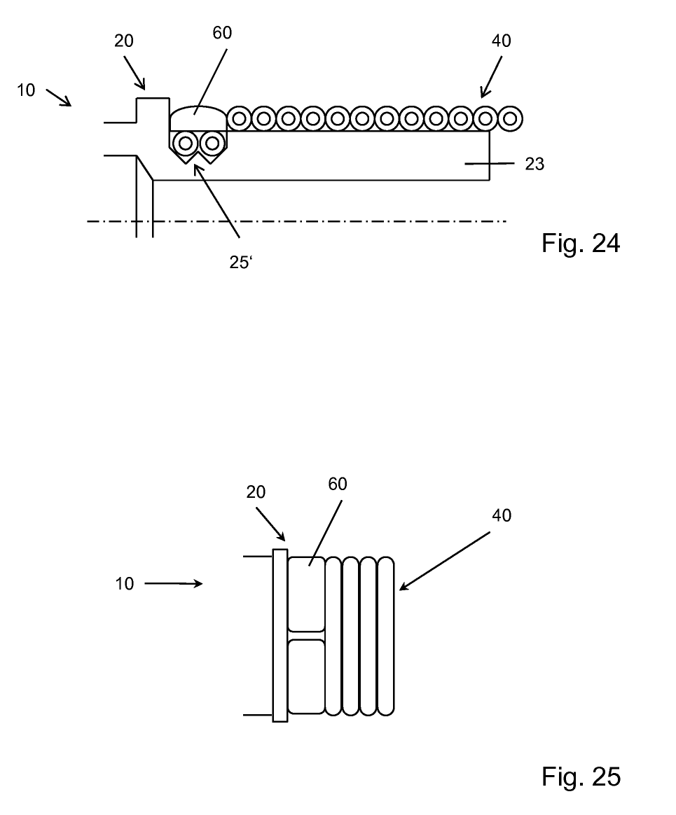

The following FIGS. 17, 19-21, 22 and 24 in each case illustrate a sectional enlargement of a stimulation electrode 20, which is embodied according to one embodiment. The sectional enlargement thereby includes that area, which is identified in FIG. 1 with the character I. FIGS. 17 and 18 in each case illustrate the medical electrode system 10 according to one embodiment in a sectional drawing and in a top view. A crimp ring 60 is used in the illustrated exemplary embodiment to connect the conduction coil 40 to the stimulation electrode 20. This crimp ring 60 can be plastically deformed. It is thus possible for the crimp ring 60 to be used to press the connection area 42 of the conduction coil 40 into the coupling element 25″ in a positive manner. It is possible in this embodiment alternative for the connection area 42 of the conduction coil 40 to be plastically deformed or also to not be plastically deformed. What is important is only that a plastic deformation of the crimp ring 60 takes place. To fix the crimp ring 60, a welding point 80 can generate a connection between the areas of the cut-open ring by means of material engagement, as is clarified in FIG. 18.

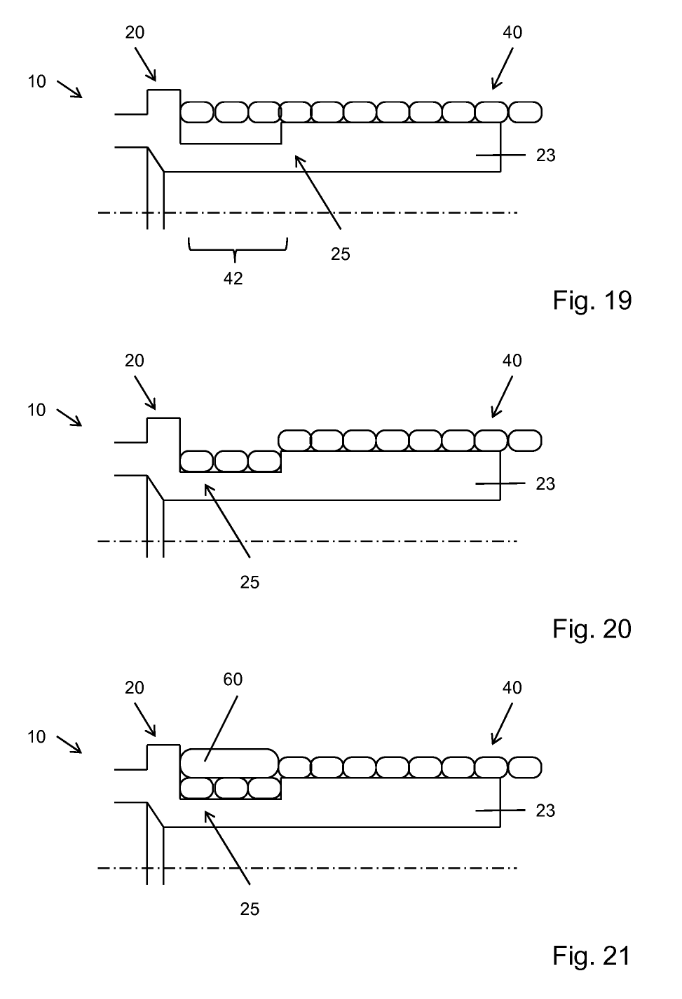

FIGS. 19-21 clarify the method according to one embodiment for connecting the conduction coil 40 to the stimulation electrode 20. For this purpose, the conduction coil 20 is slid onto the end area 23 of the stimulation electrode. In this state, the connection area 42 is not yet engaged with the coupling element 25. FIG. 20 illustrates that the connection area 42 is engaged with the coupling element 25 in a positive manner after a plastic deformation. In this case, the plastic deformation of the connection area 42 results in said connection area to be pressed into the coupling element 25. Another crimp ring 60 can then be slid across the connection area 42 of the conduction coil 40 as protection, as is illustrated in FIG. 21. The plastic deformation of the crimp ring 60 thereby ensures that it continues to exert a force onto the connection area 42 of the conduction coil 40.

The figure pairs 22, 23 and 24, 25 illustrate in each case further embodiments of the coupling element according to one embodiment including a connection area 42 of the conduction coil 40, which is pressed in each case. A crimp ring 60 was used in each case, which was deformed irreversibly such that the crimp ring 60 presses the connection area 42 of the conduction coil 40 into the groove-like coupling element 25, 25′. This pressing can, but must not lead to a plastic deformation of the connection area 42 of the conduction coil 40.

Although specific embodiments have been illustrated and described herein, it will be appreciated by those of ordinary skill in the art that a variety of alternate and/or equivalent implementations may be substituted for the specific embodiments shown and described without departing from the scope of the present invention. This application is intended to cover any adaptations or variations of the specific embodiments discussed herein. Therefore, it is intended that this invention be limited only by the claims and the equivalents thereof

1. A stimulation electrode comprising:
a base body;
wherein the base body encompasses a top area and an end area;
characterized in that the end area encompasses a groove-like, radially revolving coupling element, into which a connection area of a conduction coil can be pressed by means of plastic deformation.

2. The stimulation electrode according to claim 1, characterized in that the end area is embodied in a cylindrical manner

3. The stimulation electrode according to claim 1, characterized in that a bottom area of the coupling element is contoured so as to attain at least an area by area guiding of at least one loop of the conduction coil in the coupling element.

4. The stimulation electrode according to claim 1, characterized in that the bottom area of the coupling element encompasses a sawtooth-like cross section.

5. The stimulation electrode a according to claim 1, characterized in that the stimulation electrode encompasses at least one from the group comprising tantalum, niobium, titanium, platinum, and a TaNbW alloy.

6. A conduction coil for a medical electrode system comprising:
a conduction area; and
a connection area;
wherein the connection area connects to the conduction area and the connection area can be pressed into a coupling element of a stimulation electrode by means of plastic deformation.

7. The conduction coil according to claim 6, characterized in that the conduction coil encompasses at least one from the group comprising MP-35, MP-35N and DFT.

8. The conduction coil according to claim 6, characterized in that the connection area comprises one or a plurality of loops of the conduction coil.

9. A medical electrode system comprising:
a conduction coil; and
a stimulation electrode;
wherein the stimulation electrode encompasses a base body comprising a top area and an end area;
characterized in that the end area encompasses a groove-like, radially revolving coupling element;
wherein the conduction coil encompasses a connection area on the end side and which is engaged in a positive manner with the coupling element after a plastic deformation of the connection area.

10. The medical electrode system according to claim 9, characterized in that the conduction coil and the stimulation electrode are formed from metals comprising a different melting temperature from the group consisting of the elements Pt, Pd, Ag, Au, Nb, Ta, Ti, Zr, W, V, Hf, Mo, Co, Cr, Ni, Ir, Re, Ru and from alloys on the basis of at least one of these elements, and that the metal of the conduction coil encompasses a lower melting temperature than the metal of the stimulation electrode.

11. The medical electrode system according to at claim 9, characterized in that a crimp ring presses the connection area into the coupling element at least area by area, and that the connection area is engaged with the coupling element in a positive manner after a plastic deformation of the crimp ring.

12. A use of a crimp ring for connecting a conduction coil and a stimulation electrode.

13. The use of a crimp ring according to claim 12, characterized in that the crimp ring plastically deforms a connection area of the conduction coil.

14. The use of a crimp ring according to claim 12, characterized in that the crimp ring encompasses at least one from the group comprising tantalum, titanium, platinum, and MP-35.

15. A method for connecting a conduction coil to a stimulation electrode, characterized in that:
a connection area of the conduction coil is pressed into a groove-like coupling element of the stimulation electrode by means of plastic deformation.

16. The method according to claim 15, characterized in that the connection area of the conduction coil is pressed into a groove-like coupling element of the stimulation electrode by means of a crimp ring.

17. The method according to claim 15, characterized in that an end of the conduction coil and/or an end area of the crimp ring is welded.

18. The method according to claim 15, characterized in that at least two loops of the connection area are welded at least area by area.
